(19) United States
(12) Patent Application Publication
(10) Pub. No.: US 2006/0198908 A1
(43) Pub. Date: Sep. 7, 2006

(54) Ginseng preparation using vinegar and process for thereof

(76) Inventor: Sung Kwon Ko, Seoul (KR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/568,682

(22) PCT Filed: Aug. 18, 2003
(86) PCT No.: PCT/KR03/01660

§ 371 Date: Feb. 17, 2006

(51) Int. Cl. A61K 36/254; A61K 36/258

(52) U.S. Cl. 424/728

(57) ABSTRACT

The present invention relates to a ginseng preparation using vinegar and a process for preparing the same, more particularly to a ginseng preparation comprising high concentrations of ginsenosides (Rg₃, Rg₅, and Rh₁), which are generated by heat and exist only small amounts in red ginseng comprising various organic acids of vinegar, including citric acid, which is prepared by adding vinegar of pH 2 to 4 to ginseng, heat-extracting the same for 0.5 to 24 hours, and a method for preparing the ginseng preparation.

### BACKGROUND OF THE INVENTION

1. Field of the Invention

The present invention relates to a ginseng preparation using vinegar and a process for preparing the same, and more particularly to a ginseng preparation comprising high concentration of ginsenosides (Rg₃, Rg₅, and Rh₁) which are generated by heat and exist only small amounts in red ginseng and various organic acids of vinegar including citric acid, prepared by adding vinegar of pH 2 to 4 to ginseng, heat-extracting the same for 0.5 to 24 hours, and a method for preparing the ginseng preparation.

2. Description of the Related Art

Korean ginseng (Panax ginseng) is listed as a fine quality medicinal herb in Shennong Benaojing, a representative Chinese herbal dictionary. It has a sweet taste, is slightly warm and known effective in maintaining lungs and spleen healthy. It is also one of the specialties representing Korea. Korean ginseng contains more than 30 different kinds of ginseng saponins including ginsenoside Rb₂, which has an antidiabetic activity; polyacetylenes which have anticancer activities; phenolic compounds which have antioxidant activities; ginseng proteins which have radioprotective activities; and acidic polysaccharides, which have immune controlling activities. Further, it contains relatively large amount of phenolic compounds, polyacetylenes, and acidic polysaccharides as compared to that of American ginseng (Panax quinquefolium), and thus it is believed to have a stronger physiological activity. The ginseng saponin, which is known as the main pharmacological component of Korean ginseng, is called ‘ginsenoside’. The Shibata Group of Tokyo University has identified its chemical structure. Korean ginseng contains more than 30 different kinds of ginseng saponins, far more than those of American ginseng (14 kinds) and Sanqi ginseng (Panax notoginseng) (15 kinds). Ginsenosides are classified as protopanaxadiols and protopanaxatriols. The main component of the protopanaxadiol is ginsenoside Rb₁, and it is known to suppress the activity of central nervous system. The main component of the protopanaxatriols is ginsenoside Rg₁. It is known to excite CNS simulatory activity, and is deeply involved in the adaptogen activity of Korean ginseng.

Since the protopanaxadiol/protopanaxatriol (diol/triol) ratio and the ginsenoside Rb₁/ginsenoside Rg₁ (Rb₁/Rg₁) ratio of Korean ginseng are 1.96 and 3.14, respectively, it has a more balanced, sedative and invigoration of energy when compared to those of American ginseng, whose diol/triol ratio and Rb₁/Rg₁ ratio are 2.48 and 25.96, respectively. Therefore, Korean ginseng is believed to be one of the best tonic agents for modern people, with an improved invigoration activity and a tranquilizing activity, having a balanced ratio of ginsenoside Rb₁ and ginsenoside Rg₁.

Pharmacological activities of Korean ginseng, identified so far, are enhancement of the cardiac function and blood vessels and blood vessels; improvement of blood circulation; prevention of arteriosclerosis and hypertension; reinforcement of gastrointestinal regulatory functions; improvement of liver functions; release of hangover, anti-fatigue and anti-stress activities; prevention of aging; cognition improvement; anti-inflammatory activities; treatment of allergic diseases; treatment of women's diseases and diabetes; radioprotective activities; vital enhancement; anti-tumor activities; inhibition of lipid peroxidation; facilitation of wound healing; immune boosting activities; inhibition of AIDS virus proliferation; facilitation of protein syntheses etc.

Red ginseng (Ginseng Radix Ruba) refers to a steam-dried ginseng obtained from a garden by digging-up. White ginseng (Ginseng Radix Alba) refers to a natural-dried ginseng by sunlight after removing the peels and root hairs. And, fine ginseng root (Ginseng Radix Palba) refers to natural-dried root hairs. Particularly, the red ginseng is known to contain ginsenosides Rg₃, Rg₅, Rh₁, which are generated by heat and exist only in small amounts, are known to have activities such as cancer prevention, cancer propagation inhibition, blood pressure decrease, and antioxidation, thus drawing much attention.

While Korean ginseng has long been recognized as a brand with premium quality, it only shares about 3% of the global market. To rejuvenate the ginseng industry, it appears that the development of high value added ginseng products is necessary. Nevertheless, the ginseng preparations and red ginseng preparations introduced so far have been largely simple tonic agents because of their simple extraction process. To produce ginseng preparation products with improved functional specialties, it is essential to develop ginseng preparations comprising high concentration of physiologically active and safety-proven functional substances.

As one of such efforts to overcome one of the foregoing problems, attempts have been made to develop a ginseng preparation with a high concentration of ginsenoside Rg₃, which is known to have a superior physiological effect.

According to a Shibata's report published in 1966, prosapogenin [20(R & S)-ginsenoside Rg₃] is obtained by hydrolyzing saponin with a weak acid such as acetic acid. In this process, only the glucoside bond at C-20 (ginsenosides Rb₁, Rb₂, Rc, and Rd) was hydrolyzed and thus the process ended up with only producing a standard substance. Meanwhile, there are still other lines of studies attempting physical treatment at high-temperature or biochemical treatment using an enzyme to obtain a ginseng preparation comprising a high concentration of ginsenoside Rg₃.

In the high-temperature treatment, ginseng is heat-treated at high temperature to augment its efficacy. That is, ginseng is heat-treated at 120 to 180° C. for 0.5 to 20 hours, so that the ginsenoside ratio [(Rg₃+Rg₅)/(Rc+Rd+Rb₁+Rb₂) becomes larger than 1 to prepare a processed ginseng product or a drink composition comprising the same (Korean Patent No. 192678). An example for the biochemical treatment is to prepare a rare anticancer saponin (Rh₁, Rh₂) by hydrolyzing saccharide groups of ginseng saponin with saponin glucoside hydrolases (Korean Patent No. 329259), by which Sun Ginseng and Shin (the Almighty) Ginseng products are manufactured and released on the market. Although this method improves the efficacy of ginseng, it requires a long manufacturing period or specially designed processing equipments such as a high-pressure heater. Especially, since it requires a heat-treatment at a temperature higher than that used in the conventional processes, there is a great risk that ginseng may be charred during mass process.

The patents “Method for preparing ginsenoside Rg₃ and/or Rg₅ (Korean Patent No. 228510)” and “Vasodilator composition (Korean Patent No. 201585)” argue that effective ingredients such as Rg₃ can be obtained by acid treatment using dilute mineral acid or low grade organic acids, such as acetic acid, tartaric acid, and oxalic acid, under a mild condition, as in the heat treatment at high-temperature. However, it was difficult to obtain a ginseng preparation comprising Rg₃ and Rg₅ with acid treatment using low grade organic acids, such as acetic acid and citric acid, and also the resulting product contained a relatively large amount of impurities as identified by the component analysis.

Vinegar is classified into brewing vinegar, which is prepared by fermenting grains, fruit wines, and other alcoholic liquors; and synthetic vinegar, which is prepared by diluting glacial acetic acid or acetic acid with water (Food Code). Since vinegar has a sour taste, it stimulates the palate and promotes appetite. The sour ingredients are inorganic acids and organic acids.

Vinegar is prepared by using acid-resistant bacteria that grow fast and produce vinegar in high yield, such as Acetobacto aceti, Acetobacto acetosus, Acetobacto shuzenbachii, and Acetobacto pasteurianum, by static culture method, fast vinegar brewing method, deep fermentation method, etc.

Vinegar's effect has long been the subject of many researches. According to Dr. Krebs and Dr. Lipman (1953), vinegar releases fatigue and clears turbidity in urines within 2 hours after drinking. When people become tired due to excessive physical or mental work, lactic acid is accumulated in peoples' bodies, which then causes to promote aging process. Vinegar prevents generation of lactic acid or removes it.

In 1964, Dr. Bloch of US and Dr. Lynen of West Germany won the Nobel Prize for the theory that acetic acid in conjunction with other vinegar ingredients (citric acid, proteins, various vitamins, and minerals) are involved in producing adrenal cortical hormone. As such, various ingredients of vinegar help to prevent the generation of lactic acid or remove it, and to generate adrenal cortical hormone.

The nutritional characteristics and values of vinegar are often cited through Dr. Krebs' theory of Krebs cycle. The Krebs cycle or the TCA cycle illustrates the degradation of nutrients in our bodies. Carbohydrates and fats are digested to pyruvic acid. The pyruvic acid is metabolized to citric acid, and the citric acid is metabolized to various acids, and ultimately to water and carbon dioxide. In this process, the heat generated as a result is used for various activities. If the Krebs cycle proceeds well in a person, he will be able to stay healthy. However, if he is fatigued or overly stressed, the pyruvic acid turns into lactic acid, a representing product produced as a result of fatigue called ‘fatigue material’. Acetic acid or citric acid is absorbed by the body and metabolized, which then facilitates intestinal metabolism and releases fatigue materials like lactic acid.

Blood transfers nutrients and waste materials to and from various body parts. 92% of human blood consists of water while the remaining 8% consists of amino acid, fatty acid, glucose, various vitamins, and inorganic substances. Among the constituents of blood, inorganic materials, such as calcium, potassium, sodium, magnesium, and phosphate, maintain the alkalinity of blood, and proteins or carbohydrate metabolites maintain the blood acidity. Since these materials maintain the blood acidic and are strongly caustic, they are known to induce stomach ulcer, cystitis, constipation, etc., if remained inside the body. These hazardous materials can be removed by two different ways. One is to neutralize or inactivate them with inorganic materials such as calcium, and the other is to decompose them into water and carbon dioxide. Vinegar is known to be of great assistance to the latter process.

Since vinegar is simply used for its sour taste and flavor, its effective ingredients such as citric acid are hardly taken into consideration. There have been inventions combining vinegar and ginseng, e.g., “Method for preparing red ginseng vinegar by mixing ginseng or red ginseng with vinegar (Korean Patent No. 244849)” and “Method for preparing ginseng vinegar (Korean Patent No. 344949)”. However, the main purposes of these inventions were to prepare vinegar by mixing a small amount of ginseng or red ginseng with vinegar, and thus they are not related to the present invention which teaches the method of extracting specific ingredients from ginseng by using vinegar.

### SUMMARY OF THE INVENTION

The inventors of the present invention have made various efforts to prepare a ginseng preparation comprising high concentrations of functional materials such as ginsenoside Rg₃. In doing so, they realized that a ginseng preparation comprising high concentration of ginsenosides Rg₃, Rg₅, and Rh₁, and comprising citric acid of vinegar by adding vinegar of pH 2 to 4 to ginseng, and heat-extracting it for 0.5 to 24 hours.

Accordingly, it is an object of the present invention to provide a method for preparing a ginseng preparation comprising 5 to 100% of ginsenoside Rg₃, which has a significantly enhanced medicinal effect, with reference to the total combined ginsenosides of Rb₁, Rb₂, Rc, Rd, Re, Rf, Rg₁, and Rg₃ at a relatively low cost.

### DETAILED DESCRIPTION OF THE PREFERRED EMBODIMENTS

The present invention is characterized by a method for preparing a ginseng preparation comprising 5 to 100% of ginsenoside Rg₃ with reference to the total combined ginsenosides of Rb₁, Rb₂, Rc, Rd, Re, Rf, Rg₁, and Rg₃, and comprising 1 to 15% of (Rg3+Rg5+Rh1), by adding vinegar of pH 2 to 4 to ginseng or ginseng extract, and heat-extracting it for 0.5 to 24 hours.

Hereunder is given a more detailed description of the present invention.

The present invention relates to a method for preparing a new-version of ginseng preparation comprising high concentration of ginsenosides Rg₃, Rg₅, and Rh₁, and comprising citric acid of vinegar.

Therefore, the present invention is characterized in that it can maximize effective ingredients and contents of rare ingredients of ginseng with vinegar, enhance and support the effects of ginseng with effective ingredients of vinegar.

Hereunder is given a more detailed description of the method for preparing a ginseng preparation according to the present invention.

About 5 to 15 equivalents of vinegar of pH 2 to 4 is added to ginseng or ginseng extract, and it is heat-extracted at 70 to 150° C. for 0.5 to 24 hours to obtain a ginseng preparation. If it is heated at a temperature below 70° C., a very small amount of effective ingredient is obtained from the final product. Otherwise, if it is heated above 150° C., the content of the effective ingredient decreases and the processing becomes difficult. If the heating time is less than 0.5 hour, a very small amount of effective ingredient is obtained from the final product. In contrast, if it exceeds 24 hours, the ginsenosides Rg₃ and Rg₅ of the final product may be decomposed. The final extract according to the present invention may have liquid, powder, or any other forms.

If vinegar of pH 2.0 to 3.0 is added to ginseng or ginseng extract, and if it is extracted at 90 to 120° C. for 2 to 24 hours, a ginseng preparation comprising 50 to 100% of ginsenoside Rg₃ with reference to the total combined ginsenosides of Rb₁, Rb₂, Rc, Rd, Re, Rf, Rg₁, and Rg₃ is obtained. This ginseng preparation is useful for improving blood circulation, treating erectile dysfunction, releasing fatigue, and treating hypertension, arteriosclerosis, antithrombosis, and cerebral apoplexy. Also, vinegar of pH 2.0 to 3.0 is added to ginseng or ginseng extract and extracted at a temperature below 70 to 90° C. for 0.5 to 6 hours; or if vinegar of pH 3.0 to 4.0 is added to ginseng or ginseng extract and extracted at 90 to 120° C. for 0.5 to 6 hours, a ginseng preparation comprising 5 to 50% of ginsenoside Rg₃ with reference to the total combined ginsenosides of Rb₁, Rb₂, Rc, Rd, Re, Rf, Rg₁, and Rg₃ is obtained. This ginseng preparation is useful for preventing hypertension, arteriosclerosis, antithrombosis, and cerebral apoplexy, and for improving brain functions.

Any part of ginseng can provide the proposed effects of the present invention. That is, overground or underground parts of the genus Panax plants, including Korean ginseng (Panax ginseng), American ginseng (Panax quinquefolium), Sanqi ginseng (Panax notoginseng), Japanese ginseng (Panax japonicum), and Vietnamese ginseng (Panax vietnamensis), e.g., fine ginseng root, white ginseng, red ginseng, fresh ginseng, taeguk ginseng (boil-dried ginseng), ginseng leaves, and ginseng fruits, processed or unprocessed can be used in the present invention. This was identified through repeated experiments.

If required, ginseng can be processed to ginseng extract by the known methods. That is, ginseng is extracted with water, low grade alcohols (e.g., methanol, ethanol, etc.), low grade ketones (e.g., acetone, methyl ethyl ketone, etc.), supercritical fluids, or mixture thereof, and then concentrated. Then, the concentrate is dried to remove the solvent to obtain fluid or powdery ginseng extract.

Since vinegar prevents generation of lactic acid, a fatigue material, or removes it, facilitates metabolism, and generates adrenal cortical hormone, a ginseng preparation of the present invention, which comprises over 3% of citric acid of vinegar, enhances and aids the ginseng's effective ingredients. Vinegar is not particularly limited to those listed in the present invention, but practically any edible vinegar, such as brewing vinegar or any fermented edible vinegar can be used in the present invention. For the brewing vinegar, grain vinegar like rice vinegar, brown rice vinegar, malt vinegar, and wine lees vinegar, or fruit vinegar such as persimmon vinegar, cider vinegar, wine vinegar, pear vinegar, citrus vinegar, strawberry vinegar, and plum vinegar can be used.

A ginseng preparation of the present invention comprises not only 5 to 100% of ginsenoside Rg₃ with reference to the total combined ginsenosides of Rb₁, Rb₂, Rc, Rd, Re, Rf, Rg₁, and Rg₃, but also more than 3% of citric acid, and therefore offers superior pharmacological effects. This ginseng preparation is useful for improving blood circulation, treating erectile dysfunction, releasing fatigue, treating and preventing hypertension, arteriosclerosis, antithrombosis, and cerebral apoplexy, and improving brain functions.

The ginseng preparation of the present invention comprises 5 to 100% of ginsenoside Rg₃ with reference to the total combined ginsenosides of Rb₁, Rb₂, Rc, Rd, Re, Rf, Rg₁, and Rg₃, and 1 to 15% of (Rg₃+Rg₅+Rh₁), and therefore it offers superior pharmacological effects. Especially, since it comprises high concentration of Rg₃ (0.5 to 7.5%), Rg₅ (0.1 to 4.0%), and Rh₁ (0.2 to 3.5%), it offers superior medicinal effects. Also, since it comprises over 3% of citric acid, it enhances the pharmacological effects.

As such, the ginseng preparation of the present invention can be extracted from ginseng at a low temperature using vinegar. And, citric acid and other various organic acids, including acetic acid, of vinegar comprised in the ginseng preparation improve and enhance its pharmacological effects.

The ginseng preparation of the present invention further comprises amino acids, vitamins, and the like.

When administering the ginseng preparation of the present invention for clinical purpose at once or 2 to 3 times daily, 1 to 50 mg per kg of body weight per day is recommended. However, a specific dose may be applied depending on the chemicals to be included, body weight, age, sex, health condition, and diet of the subject, administering time, administering method, excretion rate, medicines added, and severity of disease.

The ginseng preparation of the present invention can be administered by injection or orally. A preparation for injection can be prepared using an appropriate dispersing agent, wetting agent, or emulsifying agent, e.g., aqueous or oily suspension for sterile injection, according to the known methods. Examples of usable solvents include water, Ringer's solution, and isotonic NaCl solution, and sterile oleaginous vehicle can be used for the solvent or suspension medium. Any irritation-less oleaginous vehicle including mono- and di-glyceride can be used for this purpose, and fatty acids such as oleic acid may be used for a preparation for injection. A preparation for oral administration can be prepared in the form of capsules, tablets, pills, powders, granules, and liquids. Particularly, capsules, tablets, and liquids are useful. Preferably, tablets and pills are prepared in the enteric-coating form. Solid and liquid type preparations are prepared by mixing the active ingredient with a carrier selected from more than one inert diluents like sucrose, lactose, and starch, lubricants such as magnesium stearate and talc, disintegration supporting agents such as Calcium CMC, binding agents, flavoring agents, antiseptics like sodium benzoate, sweeteners like sucrose or fructose, and surfactants. To be more specific, a capsule can be prepared by adding 7:3 ratio of starch and lactose to the active ingredient as an excipient, and adding less than 3% of magnesium stearate and talc to increase the fluidity. A tablet can be prepared by adding 7:3 ratios of starch and lactose as an excipient, a binding agent, and Calcium CMC, a disintegration supporting agent, to the active ingredient. A liquid medicine can be prepared by adding a fruit-flavoring agent, sodium benzoate as an antiseptic, sucrose or fructose as a sweetener, and a surfactant.

Hereinafter, the present invention is described in more detail through Examples and Experimental Examples. However, the following Examples and Experimental Examples are only for the understanding of the present invention, and the present invention is not limited by the following Examples and Experimental Examples.

### PREPARATION EXAMPLE

### Preparation of Ginseng Extract

50 g of fine ginseng root and 250 mL of 95% ethanol (spirituous) put in a sealed container was extracted for 4 times in a 76° C. of water bath for 4 hours, and then filtered. Thus obtained ginseng extract was vacuum-dried.

### Example 1

An amount of 10 volumes of brewing vinegar (pH 2.90) was added to 50 g of tiny ginseng, and then extracted once at 100° C. for 2 hours. The remaining solution was condensed under reduced pressure and freeze-dried to obtain a brownish extract.

### Example 2

An amount of 10 volumes of brewing vinegar (pH 2.90) was added to 50 g of fine ginseng root, and then extracted once at 100° C. for 24 hours. The remaining solution was condensed under reduced pressure and freeze-dried to obtain a brownish extract.

Instead of tiny sized ginseng, white ginseng, red ginseng, fresh ginseng, taeguk ginseng, ginseng leaves, ginseng fruits, or extracts thereof may be used.

### Example 3

An amount of eight volumes of brewing vinegar (pH 2.47) was added to 10 g of ginseng extract, and then reacted at 80° C. for 3 hours. Then, it was filtered and vacuum-dried to obtain a brownish extract. The extract was analyzed by the HPLC method.

### Example 4

An amount of eight volumes of brewing vinegar (pH 2.47) was added to 10 g of ginseng extract, and then reacted at 90° C. for 0.5 hour. Then, it was filtered and vacuum-dried to obtain a brownish extract. The extract was analyzed by the HPLC method.

### Example 5

An amount of eight volumes of brewing vinegar (pH 2.47) was added to 10 g of ginseng extract, and then reacted at 90° C. for 3 hours. Then, it was filtered and vacuum-dried to obtain a brownish extract. The extract was analyzed by the HPLC method.

### Example 6

An amount of eight volumes of brewing vinegar (pH 3.45) was added to 10 g of ginseng extract, and then reacted at 90° C. for 3 hours. Then, it was filtered and vacuum-dried to obtain a brownish extract. The extract was analyzed by the HPLC method.

### Example 7

An amount of eight volumes of brewing vinegar (pH 3.45) was added to 10 g of ginseng extract, and then reacted at 90° C. for 6 hours. Then, it was filtered and vacuum-dried to obtain a brownish extract. The extract was analyzed by the HPLC method.

### Example 8

An amount of eight volumes of brewing vinegar (pH 3.45) was added to 10 g of ginseng extract, and then reacted at 120° C. for 6 hours. Then, it was filtered and vacuum-dried to obtain a brownish extract. The extract was analyzed by the HPLC method.

### Example 9

An amount of eight volumes of brewing vinegar (pH 2.27) was added to 10 g of ginseng extract, and then reacted at 90° C. for 6 hours. Then, it was filtered and vacuum-dried to obtain a brownish extract. The extract was analyzed by the HPLC method.

### Comparative Example 1

An amount of ten volumes of distilled water was added to 50 g of fine ginseng root, and then extracted once at 100° C. for 2 hours. The remaining solution was concentrated under reduced pressure and freeze-dried to obtain a brownish extract.

### Comparative Example 2-1

An amount of eight volumes of citric acid solution (pH 5.02) was added to 10 g of ginseng extract, and then reacted at 90° C. for 3 hours. Then, it was filtered and vacuum-dried to obtain a brownish extract. The extract was analyzed by the HPLC method.

### Comparative Example 2-2

An amount of eight volumes of glacial acetic acid (pH −0.27) was added to 10 g of ginseng extract, and then reacted at 90° C. for 3 hours. Then, it was filtered and vacuum-dried to obtain a brownish extract. The extract was analyzed by the HPLC method.

### Comparative Example 3-1

An amount of eight volumes of persimmon vinegar (pH 3.42) was added to 10 g of ginseng extract, and then reacted at 60° C. for 6 hours. Then, it was filtered and vacuum-dried to obtain a brownish extract. The extract was analyzed by the HPLC method.

### Comparative Example 3-2

An amount of eight volumes of citric acid solution (pH 5.00) was added to 10 g of ginseng extract, and then reacted at 60° C. for 6 hours. Then, it was filtered and vacuum-dried to obtain a brownish extract. The extract was analyzed by the HPLC method.

### Comparative Example 3-3

An amount of eight volumes of glacial acetic acid (pH −0.27) was added to 10 g of ginseng extract, and then reacted at 60° C. for 6 hours. Then, it was filtered and vacuum-dried to obtain a brownish extract. The extract was analyzed by the HPLC method.

### Comparative Example 4-1

An amount of eight volumes of citric acid solution (pH 5.02) was added to 10 g of ginseng extract, and then reacted at 90° C. for 6 hours. Then, it was filtered and vacuum-dried to obtain a brownish extract. The extract was analyzed by the HPLC method.

### Comparative Example 4-2

An amount of eight volumes of glacial acetic acid (pH −0.27) was added to 10 g of ginseng extract, and then reacted at 90° C. for 6 hours. Then, it was filtered and vacuum-dried to obtain a brownish extract. The extract was analyzed by the HPLC method.

### Comparative Example 5-1

An amount of eight volumes of citric acid solution (pH 5.02) was added to 10 g of ginseng extract, and then reacted at 120° C. for 6 hours. Then, it was filtered and vacuum-dried to obtain a brownish extract. The extract was analyzed by the HPLC method.

### Comparative Example 5-2

An amount of eight volumes of glacial acetic acid (pH −0.27) was added to 10 g of ginseng extract, and then reacted at 120° C. for 6 hours. Then, it was filtered and vacuum-dried to obtain a brownish extract. The extract was analyzed by the HPLC method.

### Experimental Example 1

### Ginsenoside Rg₃ Analysis by HPLC

(1) Test Method

50 g of each test sample was treated 3 times with ether. The water-soluble layer was treated 3 times with water-saturated n-butanol. The n-butanol layer was condensed under reduced pressure to obtain crude saponin (Shibata method). The crude saponin was quantified by the HPLC method. The result is shown in the following Tables 1, 2, and 3.

(2) HPLC Analysis Condition:

For each ginseng saponin ingredient, a calibration curve was drawn based on 1 mg/mL (1000 ppm). Each sample was prepared to a concentration of 10 mg/mL (10000 ppm). The HPLC condition is as follows:
HPLC: Gilson 305 system
Column: μ-Bondapak C18 (Waters, 3.9×150 mm)
Detector: Gilson 118 UV/detector
Temperature: room temperature

Mobile phase: (CH₃CN, 17%→33%→60%→80%→17%)

TABLE 1
Ginsenoside contents of ginseng preparation

| | Crude | Total | Ginsenoside content (w/w %) | | | | | | | | | | | |
|---|---|---|---|---|---|---|---|---|---|---|---|---|---|---|
| Preparation | saponin (%) | saponin (%) | Rb₁ | Rb₂ | Rc | Rd | Re | Rf | Rg₁ | Rh₁ | Rg₃ | Rg₅ | Formula 1* | Formula 2** |
| Comp. | 2.78 | 5.65 | 1.853 | 0.597 | 1.125 | 0.489 | 0.550 | 0.097 | 0.142 | 0.796 | 0.000 | 0.000 | 0.796 | — |
| Example 1 | | | | | | | | | | | | | | |
| Example 1 | 1.94 | 6.14 | 0.043 | 0.058 | 0.219 | 0.108 | 0.020 | 0.137 | 0.012 | 1.253 | 1.477 | 2.813 | 5.54 | 71.22 |
| Example 2 | 2.98 | 2.82 | 0.000 | 0.000 | 0.000 | 0.019 | 0.014 | 0.009 | 0.000 | 0.505 | 0.557 | 1.712 | 2.77 | 92.99 |
| Example 3 | — | — | 1.46 | 1.10 | 1.07 | 1.07 | 1.20 | 0.12 | 0.59 | 0.43 | 0.66 | 0.13 | 1.22 | 9.07 |
| Example 4 | — | — | 1.19 | 1.24 | 1.20 | 1.15 | 1.59 | 0.17 | 0.82 | 0.46 | 0.82 | 0.12 | 1.40 | 10.02 |

*Rg₃ + Rg₅ + Rh₁
**[Rg₃/(Rb₁ + Rb₂ + Rc + Rd + Re + Rf + Rg₁ + Rg₃)] × 100

Ginsenoside contents of crude saponins obtained from the fine ginseng root extract (Comparative Example) and the ginseng preparations prepared by the Shibata method according to the present invention (Examples) were analyzed by the HPLC method. As seen in Table 1, while ginsenoside Rg₃, the specific ingredient of red ginseng, was not detected at all from the fine ginseng root extract, but high concentration of ginsenoside Rg₃ were detected from the ginseng preparations of the present invention. Particularly, the preparation of Example 1 showed the highest ginsenoside Rg₃ content of 1.477%, which corresponds to 71.22% of the total saponin contents. Further, the preparation of Example 2 also showed high ginsenoside Rg₃ content of 0.557%, which corresponds to 92.99% of the total saponin contents.

TABLE 2

| | Ginsenoside content (w/w %) | | | | | | | | | | | |
|---|---|---|---|---|---|---|---|---|---|---|---|---|
| Preparation | Rb₁ | Rb₂ | Rc | Rd | Re | Rf | Rg₁ | Rh₁ | Rg₃ | Rg₅ | Formula 1* | Formula 2** |
| Example 5 | 0.03 | 0.15 | 0.11 | 0.81 | 0.81 | 0.74 | 0.71 | 1.23 | 1.93 | 0.12 | 2.86 | 33.80 |
| Example 6 | 0.07 | 0.31 | 0.11 | 0.65 | 0.98 | 0.88 | 0.83 | 0.97 | 1.27 | 0.32 | 2.24 | 23.43 |
| Comp. Example 2-1 | 0.52 | 1.92 | 0.12 | 0.27 | 1.86 | 1.61 | 1.49 | 1.55 | 0.26 | 0.17 | 0.70 | 2.79 |
| Comp. Example 2-2 | 0.06 | 0.17 | 0.07 | 0.15 | 0.04 | 0.05 | 0.00 | 0.00 | 0.59 | 0.59 | 1.33 | 60.20 |

*Rg₃ + Rg₅ + Rh₁
**[Rg₃/(Rb₁ + Rb₂ + Rc + Rd + Re + Rf + Rg₁ + Rg₃)] × 100

As seen in Table 2, when the pH ranged from 2 to 4 (Examples 5 and 6), the Rg₃ contents increased significantly to 1.93% and 1.27%, compared to when the pH was below 2 or over 4 (Comparative Examples 2-1 and 2-2), whose Rg₃ contents were 0.26% and 0.59%, respectively. In Comparative Example 2-2, the total saponin contents were significantly lower than those in other Examples. It is speculated that the glacial acetic acid with strong acidity may prevent generation of Rg₃.

TABLE 3

| | | | | Comp. | Comp. | Comp. | Comp. | Comp. | Comp. | Comp. |
|---|---|---|---|---|---|---|---|---|---|---|
| Preparation | Example 7 | Example 8 | Example 9 | Example 3-1 | Example 3-2 | Example 3-3 | Example 4-1 | Example 4-2 | Example 5-1 | Example 5-2 |
| Rg₃ | 2.36 | 2.28 | 5.93 | 0.19 | 0.17 | 0.31 | 0.43 | 0.63 | 0.59 | 0.25 |
| Rg₅ | 0.40 | 0.42 | 1.18 | 0.05 | 0.06 | 0.31 | 0.13 | 0.77 | 0.08 | 0.22 |
| Rh₁ | 0.77 | 1.53 | 0.78 | 0.64 | 0.33 | 0.16 | 0.40 | 0.21 | 1.02 | 0.00 |
| Formula 1* | 3.53 | 4.23 | 7.89 | 0.88 | 0.56 | 0.78 | 0.96 | 1.61 | 1.69 | 0.47 |
| Formula 2** | 44.19 | 48.94 | 98.02 | 1.40 | 1.33 | 2.57 | 5.56 | 82.89 | 4.98 | 83.33 |

*Rg₃ + Rg₅ + Rh₁
**[Rg₃/(Rb₁ + Rb₂ + Rc + Rd + Re + Rf + Rg₁ + Rg₃)] × 100

As seen in Table 3, when the reaction was performed at a temperature below 70° C. (Comparative Examples 3-1, 3-2, and 3-3) the Rg₃ contents were as low as 0.19%, 0.17%, and 0.31%, respectively. However, when the reaction was performed at a temperature above 90° C. (Examples 7 and 8), the Rg₃ contents increased. When the pH ranged from 2 to 4, there were no significant difference in Rg₃ content at 90° C. and 120° C. However, when the pH was higher than 4, the Rg₃ content increased in proportion to the increase in temperature. This implies that the reaction temperature can play a role to some extent in generation of Rg₃ up to a certain level of acidity.

Therefore, a ginseng preparation comprising a high concentration of Rg₃ can be prepared by adjusting the acidity (pH) and the amount of vinegar at a reaction temperature of 70 to 150° C. and a reaction time of 0.5 to 24 hours.

### Experimental Example 2

### Citric Acid Contents of Ginseng Preparation

For the ginseng preparations prepared from Examples 1 to 9, and red ginseng (control group), citric acid contents were analyzed by the following method. The result is shown in the following Table 4.

HPLC Analysis Condition:

For each citric acid ingredient, a calibration curve was drawn based on 1 mg/mL (1000 ppm). Each sample was prepared to a concentration of 10 mg/mL (10000 ppm). The HPLC condition is as follows:
HPLC: Gilson 305 system
Column: μ-Bondapak C18
Detector: UV 210 nm

Mobile phase: (Pump: 5 mM H₂SO₄)—Isocratic acid

TABLE 4

| Preparation | Citric acid (%) |
|---|---|
| Red ginseng | 0 |
| Example 1 | 4.725 |
| Example 2 | 4.586 |
| Example 3 | 3.522 |
| Example 4 | 3.467 |
| Example 5 | 3.234 |
| Example 6 | 3.758 |
| Example 7 | 3.445 |
| Example 8 | 3.562 |
| Example 9 | 3.685 |

As seen in Table 4, the citric acid contents of Examples were higher than 3%, much greater than that in red ginseng, due to the component of the vinegar contained in the ginseng preparations.

### Experimental Example 3

From the ingredient analysis of the ginseng preparations prepared in Examples, the following amino acids and vitamins were identified.

<Test result>

| Vitamin B₁ (mg/100 g) | Undetected |
|---|---|
| Vitamin B₂ (mg/100 g) | 1.1 |
| Lysine (mg/100 g) | 73.8 |
| Isoleucine (mg/100 g) | 107.9 |
| Tryptophane (mg/100 g) | 210.3 |
| Histidine (mg/100 g) | 173.1 |
| Arginine (mg/100 g) | 434.5 |
| Threonine (mg/100 g) | 113.4 |
| Valine (mg/100 g) | 137.1 |
| Niacin (mg/100 g) | 6.9 |
| Methionine + cysteine (mg/100 g) | 236.7 |
| Phenylalanine + tyrosine (mg/100 g) | 308.4 |

As described in detail above, a ginseng preparation comprising high concentrations of ginsenosides Rg₃, Rg₅, and Rh₁, which are functional materials generated in low yield during preparation of red ginseng, and comprising citric acid of vinegar can be easily prepared according to the present invention.

While the present invention has been described in detail with reference to the preferred embodiments, those skilled in the art will appreciate that various modifications and substitutions can be made thereto without departing from the spirit and scope of the present invention as set forth in the appended claims.

What is claimed is:

1. A method for preparing a ginseng preparation comprising 5 to 100% of ginsenoside Rg₃ with reference to the total combined ginsenosides of (Rb₁, Rb₂, Rc, Rd, Re, Rf, Rg₁, and Rg₃), and 1 to 15% of (Rg₃+Rg₅+Rh₁), prepared by adding vinegar of pH 2 to 4 to ginseng or ginseng extract, and then heat-extracting it for 0.5 to 24 hours.

2. The method for preparing a ginseng preparation according to claim 1, wherein said ginseng preparation comprises 0.5 to 7.5% of Rg₃, 0.1 to 4.0% of Rg₅, and 0.2 to 3.5% of Rh₁.

3. The method for preparing a ginseng preparation according to claim 1, wherein said heating is performed at 70 to 150° C.

4. The method for preparing a ginseng preparation according to claim 1, wherein said ginseng is overground or underground part of the genus Panax plant, or extract prepared therefrom.

5. The method for preparing a ginseng preparation according to claim 4, wherein said genus Panax plant is selected from the group consisting of Korean ginseng (Panax ginseng), American ginseng (Panax quinquefolium), Sanqi ginseng (Panax notoginseng), Japanese ginseng (Panax japonicum), and Vietnamese ginseng (Panax vietnamensis).

6. The method for preparing a ginseng preparation according to claim 4 or claim 5, wherein said overground or underground part of the genus Panax plant is selected from the group consisting of fine ginseng root, white ginseng, red ginseng, fresh ginseng, taeguk ginseng, ginseng leaves, and ginseng fruits.

7. The method for preparing a ginseng preparation according to claim 1, wherein said vinegar is brewing vinegar.

8. The method for preparing a ginseng preparation according to claim 7, wherein said brewing vinegar is grain vinegar or fruit vinegar.

9. The method for preparing a ginseng preparation according to claim 8, wherein said grain vinegar is selected from the group consisting of rice vinegar, brown rice vinegar, malt vinegar, and wine lees vinegar, and the fruit vinegar is selected from the group consisting of persimmon vinegar, cider vinegar, wine vinegar, pear vinegar, citrus vinegar, strawberry vinegar, and plum vinegar.

10. The method for preparing a ginseng preparation according to claim 1, wherein said ginseng preparation is prepared by adding vinegar of pH 2.0 to 3.0 to ginseng, and then heat-extracting it at 90 to 120° C. for 2 to 24 hours.

11. The method for preparing a ginseng preparation according to claim 1, wherein said ginseng preparation is prepared by adding vinegar of pH 2.0 to 3.0 to ginseng, and then heat-extracting it at a temperature lower than 70 to 90° C. for 0.5 to 6 hours.

12. The method for preparing a ginseng preparation according to claim 1, wherein said ginseng preparation is prepared by adding vinegar of pH 3.0 to 4.0 to ginseng, and then heat-extracting it at 90 to 120° C. for 0.5 to 6 hours.

13. A ginseng preparation prepared by any method according to claims 1 to 12, which comprises 5 to 100% of ginsenoside Rg₃ with reference to the total combined ginsenosides of (Rb₁, Rb₂, Rc, Rd, Re, Rf, Rg₁, and Rg₃).

14. The ginseng preparation according to claim 13, which comprises 1 to 15% of (Rg₃+Rg₅+Rh₁).

15. The ginseng preparation according to claim 13 or claim 14, which comprises 0.5 to 7.5% of Rg₃, 0.1 to 4.0% of Rg₅, and 0.2 to 3.5% of Rh₁.

16. The ginseng preparation according to claim 13 or claim 14, which comprises more than 3% of citric acid.

17. The ginseng preparation according to claim 13 or claim 14, which comprises 50 to 100% of ginsenoside Rg₃ with reference to the total combined ginsenosides of (Rb₁, Rb₂, Rc, Rd, Re, Rf, Rg₁, and Rg₃).

18. The ginseng preparation according to claim 13 or claim 14, which comprises 5 to 50% of ginsenoside Rg₃ with reference to the total combined ginsenosides of (Rb₁, Rb₂, Rc, Rd, Re, Rf, Rg₁, and Rg₃).
